(19) United States
(12) Patent Application Publication
(10) Pub. No.: US 2008/0294104 A1
(43) Pub. Date: Nov. 27, 2008

(54) Microcatheter Including Swellable Tip

(76) Inventor: Michel E. Mawad, (US)

Correspondence Address: INSKEEP INTELLECTUAL PROPERTY GROUP, INC, 2281 W. 190TH STREET, SUITE 200, TORRANCE, CA, 90504, US

(21) Appl. No.: 12/188,581

(22) Filed: Aug. 8, 2008

Related U.S. Application Data
(63) Continuation of application No. 10/755,249, filed on Jan. 12, 2004, now abandoned.
(60) Provisional application No. 60/439,229, filed on Jan. 10, 2003.

(51) Int. Cl. A61M 25/04

(52) U.S. Cl. 604/103.03

(57) ABSTRACT

A catheter for providing an embolic material at a desired location comprises a flexible tubular member having a proximal end and a distal end, the tubular member including at least one ring of expansible material affixed to its outer surface less than 25 mm from the distal end. The ring preferably comprises a material that expands in volume when in contact with a liquid, such as a hydrogel or hydrogel foam, or in response to an application of heat. The ring is fully expanded after between 10 and 30 minutes of contact with a liquid. The catheter may also include further including a plurality of radioopaque markers spaced along the tubular member at predetermined intervals.

The present invention relates to systems and methods for treating vascular malformations and tumors. Blood vessels will occasionally develop in a manner that can pose a health risk. Abnormal vascular connections, known as arteriovenous malformations (AVMs), may develop either as a congenital defect or as a result of iatrogenic or other trauma. An AVM may lead to a substantial diversion of blood from the intended tissue and may consequently engender a variety of symptoms, which are sometimes life-threatening.

Endovascular therapy has been used to treat AVMs and vascular tumors, including control of internal bleeding, occlusion of blood supply to the lesions, and relief of vessel wall pressure in the region of an aneurysm. Such treatments typically require the use of a catheter to deliver various embolic materials, devices, and drugs at remote locations within the body. Microcatheters, such as those shown by Engleson, “Catheter Guidewire”, U.S. Pat. No. 4,884,579 and as described in Engleson, “Catheter for Guidewire Tracking”, U.S. Pat. No. 4,739,768, allow navigation through the body's tortuous vasculature to access such remote sites as the liver or the cerebral arteries of the brain.

In some instances it may be desirable to create an endovascular occlusion at the site of the lesion. A microcatheter is typically used to place a vaso-occlusive device or agent within the lesion to block the flow of blood through a vessel and to form an embolus. Suitable microcatheters are well known in the art and include flow directed catheters such as the FlowRider® flow-directed micro catheter and over-the-wire catheters such as the Rebar® over-the-wire micro catheter.

Formation of the embolus may involve the injection of a fluid embolic agent such as microfibrillar collagen, silicone polymer beads, or polymeric resins such as cyanoacrylate, which polymerize in situ. Ideally, the embolic agent conforms to the irregular shape of the internal walls of the malformation or aneurysm. A particularly effective embolic agent is the liquid adhesive isobutylcyanoacrylate (IBCA), which polymerizes rapidly on contact with blood to form a firm mass. Another effective agent is comprises a biocompatible liquid embolic agent such as ethylene vinyl alcohol copolymer (EVOH) dissolved in dimethyl sulfoxide (DMSO). An example of such as system is sold under the trade name Onyx™. If desired, a radioopaque material such as micronized tantalum powder may be added to the polymer to provide contrast for fluoroscopy. This type of system solidifies as a result of precipitation. Precipitation is initiated when the injected solution comes into contact with an aqueous solution (e.g., blood, body fluids normal saline, water) and the solvent DMSO rapidly diffuses out of the polymer mass. The resulting precipitation of the polymer produces a spongy mass that flows into the vascular malformation.

In one common technique, the embolic material is injected into the AVM using a microcatheter. One risk with this procedure is inadvertent embolism in the parent artery due to the inability to contain the fluid agent within the AVM/lesion.

The distance and direction that any injected embolic material travels before solidifying within the AVM/lesion depends on a number of factors, including the flow rate in the vessel, the rate of injection, and the setting time of the embolic material. If blood flow in the vessel is low, if flow into the AVM is difficult, or if the catheter tip does not sufficiently obstruct the opening to the AVM, backflow may occur, with the result that the embolic material flows back around the catheter instead of into the AVM. While a small amount of backflow can be tolerated, and may even be advantageous, extensive backflow may cause undesired obstruction of other vessels and/or render removal of the catheter difficult.

Hence, in order to reduce backflow into the parent artery during injection of the embolic material, some practitioners attempt to reduce or interrupt fluid flow through the parent artery. According to one technique, an inflated balloon is placed in the artery feeding the AVM. This system had drawbacks, however, inasmuch microcatheters equipped with balloons cannot be made small enough to treat AVMs that occur in smaller vessels.

Despite advances made in various aspects of this treatment, it remains desirable to provide a system and method for providing an embolic material to an AVM without allowing undesired backflow or leakage of the embolic material past the catheter tip.

### SUMMARY OF THE INVENTION

The present invention provides a system and method for providing an embolic material to an AVM without allowing undesired backflow or leakage of the embolic material past the catheter tip. The present system includes a microcatheter that is provided with a plug of expansile material adjacent to its tip. In one preferred embodiment, the expansile material comprises a hydrogel or other material that swells or expands when it contacts the in vivo environment. In other embodiments, the expansile material comprises a substance that swells or expands when a stimulus, such as heat or a voltage potential, is applied.

### BRIEF DESCRIPTION OF THE DRAWINGS

For a more detailed understanding of the invention, reference is made to the accompanying Figures, wherein:

FIG. 1 is a schematic illustration of a catheter constructed in accordance with a preferred embodiment of the invention;

FIGS. 2 and 3 are cross-sections of the catheter of FIG. 1 taken at the juncture of the proximal portion and the distal portion and at the tip of the distal portion, respectively; and

FIG. 4 is an illustration of the catheter of FIG. 1 emplaced in a vessel and injecting an embolic material into an AVM.

### DETAILED DESCRIPTION OF THE PREFERRED EMBODIMENTS

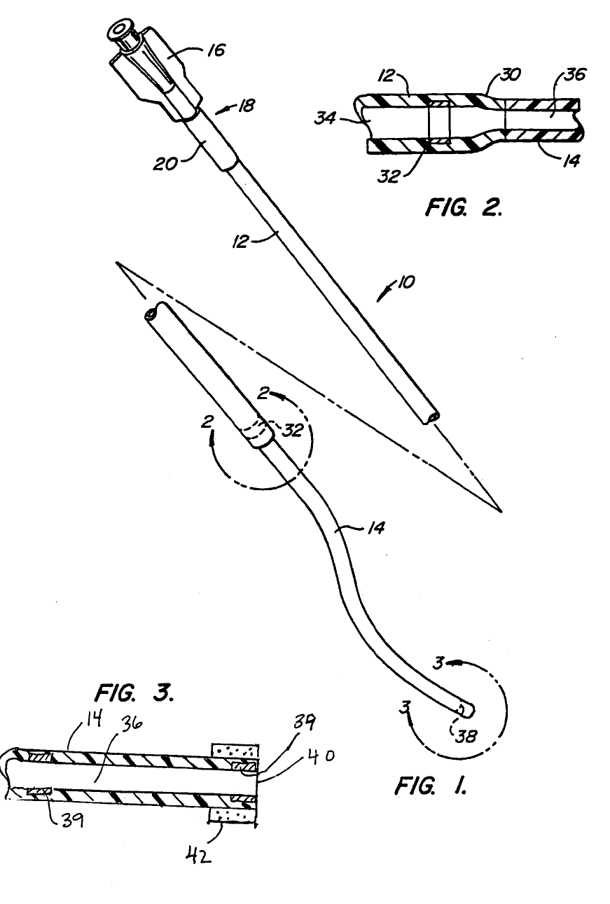

Referring initially to FIGS. 1-3, an exemplary flow-directed catheter 10 that could be used in conjunction with the principles of the present invention comprises a catheter body including a proximal body segment 12 and a distal body segment 14. A standard luer connector 16 is preferably attached to a proximal end 18 of the proximal body segment 12, and a strain relief sleeve 20 is preferably included adjacent to connector 16. As best illustrated in FIG. 2, a shoulder 30 is provided near the distal end of the proximal body segment 12, and a radiopaque marker 32 is preferably provided near the shoulder, at the transition, preferably recessed within lumen 34 of the proximal body segment 12. The inner diameter of the radiopaque marker ring 32 may be generally concentric with the lumen 34 in the proximal body segment 14, as shown, or could alternatively be concentric and aligned with the lumen 36 of the distal body segment. Additional features relating to flow directed catheters are described in U.S. Pat. No. 5,899,890, which is incorporated herein by reference.

Referring now particularly to FIG. 3, in a preferred embodiment of the invention the distal end 40 of distal body segment 14 includes at least one annular ring 42 on its outer surface. Ring 42 is preferably constructed of an expansile or expandable material. Suitable materials include, but are not limited to: hydrogels; hydrophilic polymers with or without conjugated collagen as described in U.S. Pat. No. 5,413,791, which is incorporated herein by reference; biocompatible, macroporous, hydrophobic hydrogel foams; and compressible, non-hydrophobic polymeric foam materials, such as polyurethane. A particularly preferred foam includes a water-swellable foam matrix formed as a macroporous solid comprising a foam stabilizing agent and a polymer or copolymer of a free radical polymerizable hydrophobic olefin monomer cross-linked with up to about 10% by weight of a multiolefin-functional cross-linking agent, as described in detail in. A suitable material of this type is described in U.S. Pat. Nos. 5,570,585 and 6,500,190, the disclosures of which are incorporated herein by reference. Another suitable material is a porous hydrated polyvinyl alcohol foam (PAF) gel prepared from a polyvinyl alcohol solution in a mixed solvent consisting of water and a water-miscible organic solvent, as described, for example, in U.S. Pat. No. 4,663,358, which is also incorporated herein by reference. Still another suitable material is PHEMA, as discussed in the references cited above. The expansible material can expand as a result of hydration of it molecular structure, or by the filling of its pores with liquid (blood), or both.

Alternatively, expansile ring 42 can be made of a material that expands upon application of a controlled stimulus, such as heat or a voltage potential. For example, a heat-swellable gel, foam, or resin can be heated by passing a bolus of warm fluid through catheter 10. Alternatively, ring 42 can be made of a material that expands upon application of a voltage potential.

Ring 42 is preferably sized such that its outer diameter prior to expansion is smaller than the diameter of the vessel at the point where occlusion is desired. This will ensure that the tip 40 of catheter 10 can be positioned at the desired point without becoming stuck in the vessel. Ring 42 is preferably constructed of material that is capable of swelling from its initial size to an expanded size that is large enough to occlude the vessel in which it is placed, but without causing damage to the vessel. Thus, the material from which ring 42 is constructed is preferably selected to provide an expansion ratio that corresponds to the starting and expanded sizes of ring 42.

Similarly, the material from which ring 42 is constructed is selected such that the time required for it to reach its desired expanded diameter is within a given range, preferably between about 10 and 40 minutes and more preferably between about 20 and 30 minutes. If ring 42 expands too quickly, it may become lodged in a vessel in a location other than its intended location; hence it is preferred that ring 42 expand sufficiently slowly to allow the physician to position the catheter tip at the desired location before the vessel becomes occluded. On the other hand, if the catheter tip is positioned at the desired location, the physician can simply wait for the prescribed expansion period before continuing the procedure, provided that the expansion period is not unduly long. Most practitioners can complete placement of a catheter at an intracranial location in about 5 to 15 minutes.

As shown in FIG. 3, it is preferred to position ring 42 as closely as possible to the catheter tip 40. Specifically, ring 42 is affixed to the outer surface of catheter tip between zero and 25 mm and preferably between zero and 5 mm from tip 10. The axial length of ring 42 may be any desired amount, and is preferably about 1-4 times the inside diameter of the vessel. Furthermore, in some instances it may be desired to provide a second or further ring of expansible material. In a preferred embodiment, a plurality of radiopaque markers 39 are included at predetermined distances from tip 40. Markers 39 facilitate placement of catheter tip 40, and can comprise any suitable material such as are known in the art. The plurality of the markers at predetermined distances can also be used for measurement purposes.

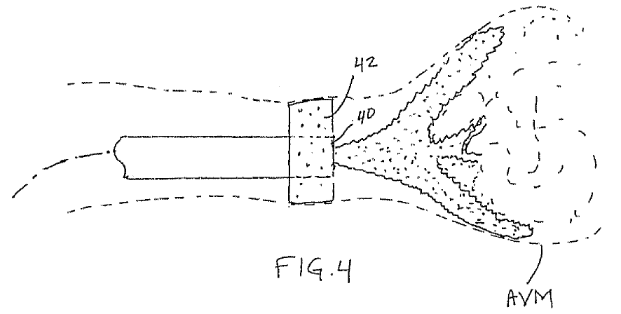

Referring now to FIG. 4, the present catheter is shown with ring 42 expanded inside a vessel and a mass of embolic material being injected from catheter tip 40 into an AVM or a tumor (vessel and AVM shown in phantom). The expanded ring 42 prevents the embolic material from flowing back along catheter 10.

When the desired amount of embolic material has been injected and it is desired to remove catheter 10 from the vessel, the catheter is pulled back from the vessel and the expansile ring 42 is detached from tip 40. The detachable expansile tip 42 remains in the feeding vessel held in place by the embolic material which would have refluxed around it.

While a preferred embodiment has been shown and described, it will be understood that various modifications could be made thereto without departing from the scope of the invention.

1. A catheter for providing an embolic material at a desired location in a vessel, comprising:
a flexible tubular member having a proximal end and a distal end, and an outer surface, said tubular member including at least one ring of expansible material affixed to said its outer surface proximate said distal end, wherein said at least one ring has an expanded size which occludes the space between the outer surface and the vessel:
an injector configured to release embolic material proximate said distal end of said flexible tubular member and into the vessel: and
wherein the embolic material and the at least one ring occlude the vessel.

2. The catheter according to claim 1, wherein said ring comprises a material that expands in volume when in contact with a liquid.

3. The catheter according to claim 2, wherein said ring comprises a hydrogel.

4. The catheter according to claim 2, wherein said ring comprises a hydrogel foam.

5. The catheter according to claim 2, wherein said ring is fully expanded after between 10 and 30 minutes of contact with a liquid.

6. The catheter according to claim 1, wherein said ring is affixed to said tubular member less than 10 mm from said distal end.

7. The catheter according to claim 1, wherein said ring is affixed to said tubular member less than 5 mm from said distal end.

8. The catheter according to claim 1, wherein said ring is affixed to said tubular member adjacent to said distal end.

9. The catheter according to claim 1, wherein said ring expands in response to an application of heat.

10. The catheter according to claim 1, further including multiple radioopaque markers spaced along the tubular member at predetermined intervals.

11. The catheter according to claim 1, wherein the embolic material contacts the at least one expansible ring after the material is released into the vessel.

12. The catheter according to claim 1, wherein the at least one ring is detachable from said tubular member while the tubular member is at the desired location within the vessel.

13. The catheter according to claim 1, wherein the at least one ring is affixed to said tubular member no greater than 25 mm from said distal end.

14. The catheter according to claim 1, wherein said at least one ring of expansible material reaches its expanded size in response to application of a controlled stimulus.

15. The catheter according to claim 1, wherein said at least one ring of expansible material reaches its expanded size in response to an in vivo environment within the vessel.

16. The catheter according to claim 1, wherein said at least one ring of expansible material reaches its expanded size in response to a combination of contact with an in vivo environment in the vessel and application of a controlled stimulus.

17. The catheter according to claim 1, wherein the combination of said at least one ring of expansible material in its expanded size and said embolic material occludes the vessel.

18. A catheter for providing an embolic material at a desired location in a vessel, comprising:
a flexible tubular member having a proximal end, a distal end, and an external surface, said flexible tubular member further having an expansible portion adjacent said distal end; and
wherein said expansible portion has a resting size and an expanded size, said resting size closely approximating said external surface of said flexible tubular member and said expanded size being sufficient to occlude the space between said flexible tubular member and the vessel; and
wherein said expansible portion is detachable from said flexible tubular member while said flexible tubular member is at the desired location in the vessel.

19. The catheter according to claim 18, further including an injector configured to inject an embolic material from said distal end of said flexible tubular member into the vessel.

20. The catheter according to claim 18, wherein said expansible portion expands from said resting size to said expanded size in response to application of a controlled stimulus.

21. The catheter according to claim 18, wherein said expansible portion expands from said resting size to said expanded size in response to contact with an in vivo environment within the vessel.

22. A catheter for providing an embolic material at a desired location in a vessel, comprising:
an elongated tubular member having a proximal end, a distal end, and a circumference, said elongated tubular member further having an expansible portion comprising an expandable material about said circumference and proximate said distal end; and
wherein said distal end is configured to permit embolic material to be placed in the vessel through said distal end;
wherein said expansible portion is configured to expand from a first position to a second position, said second position occluding the space between said circumference of said elongated tubular member and the vessel;
wherein said embolic material and said expansible portion in said second position occlude the vessel; and
wherein said expansible portion is detachable from said elongated tubular member while said elongated tubular member is at the desired location in the vessel.

23. The apparatus of claim 22, where said expansible portion is detachable from said elongated tubular member while at the desired location in the vessel.
